(19) United States
(12) Patent Application Publication
(10) Pub. No.: US 2011/0002912 A1
(43) Pub. Date: Jan. 6, 2011

(54) COMPOSITION

(75) Inventors: Gloria A. Limb, London (GB); James Fawcett, Cambridge (GB); Jean Lawrence, London (GB); Peng T. Khaw, London (GB)

Correspondence Address: BANNER & WITCOFF, LTD., 1100 13th STREET, N.W., SUITE 1200, WASHINGTON, DC, 20005-4051, US

(73) Assignees: UCL BUSINESS PLC, London (GB); CAMBRIDGE ENTERPRISE LIMITED, Cambridge (GB)

(21) Appl. No.: 12/746,109

(22) PCT Filed: Dec. 5, 2008
(86) PCT No.: PCT/GB2008/004021

§ 371 Date: Sep. 20, 2010

(30) Foreign Application Priority Data
Dec. 5, 2007 (GB) 0723825.6

(51) Int. Cl. A61K 39/395; A61K 38/47; A61K 38/48; A61K 38/51; A61K 35/12; A61P 43/00; A61P 25/00; A61P 25/16; A61P 27/02

(52) U.S. Cl. 424/130.1; 424/94.62; 424/94.67; 424/94.5; 424/93.7

(57) ABSTRACT

The present invention relates to a composition for improving stem cell integration. The present invention also relates to a method of improving stem cell integration and kits for improving stem cell integration.

The present invention relates to a composition for improving stem cell integration. The present invention also relates to a method of improving stem cell integration and kits for improving stem cell integration.

Stem cell based therapies provide hope for the treatment of numerous diseases, including diseases of the central nervous system (CNS), any solid organ (e.g., heart, liver, kidney, eyes) and muscular disorders. Stem cell therapy is based on the finding that stem cells can be used to replace diseased or damaged cells. Stem cells that are capable of differentiating into the appropriate cell type in order to replace the diseased cells are delivered to the site of the diseased or damaged cells. The stem cells functionally integrate into the tissue and differentiate into the appropriate cell type to replace the damaged or diseased cells. By doing this numerous diseases and disorders can be treated.

Unfortunately, there are a number of difficulties with stem cell therapies. In particular it is difficult to ensure that the stem cells survive, differentiate and functionally integrate into the tissue to be treated. Without suitable integration, stem cell therapy has limited value.

The present invention is particularly concerned with the restoration of sight in individuals whose retinal function has been irreversibly damaged by disease. Transplantation of neural stem cells and retinal progenitors has been extensively performed in various experimental models of retinal disease, with several studies yielding a mixed success to date (1-7). However, before this procedure can be translated into human therapies, many problems need to be solved to promote adequate cell survival, differentiation and functional integration of grafted cells into the retina. Studies involving retinal transplantation of brain-derived precursor cells into Royal College of Surgeons (RCS) rats have been shown to promote photoreceptor cell survival, but although the transplanted cells migrate to the photoreceptor layer in some instances, they do not express retinal-specific markers (8-10). It has been suggested that either specific retinal precursors are needed for functional and morphological regeneration, or that specific cues such as retinal injury are needed for appropriate migration and differentiation of transplanted cells. These views have been confirmed by observations that brain progenitors and ocular stem cells show improved, although not optimal integration and differentiation into retinal neurons when transplanted in injured retina (11;12). It has also been shown that neonate retina provides an amenable environment for stem cell transplantation (13) and that the ontogenic stage of transplanted retinal precursors determines the ability of these cells to integrate into degenerated retina (14).

Müller glial cells have shown neural regenerative ability in the postnatal retina of zebra fish, chick and rat (15-17), and a population of Müller glia with neural stem cell characteristics has been recently identified in the adult human eye (18). These cells have the potential to be used in cell based therapies to treat retinal disease, but like other stem cells used for experimental transplantation to regenerate retina, when grafted into neonate and degenerated retina they show limited migration and integration (18). Müller stem cells and their use in the treatment of visual disorders is described in International PCT application WO 2005/054447. Retinal degeneration is characterized by formation of glial scarring (19) and severe microglial activation (20-22), which may contribute to the lack of migration, integration and differentiation of transplanted stem cells. Adult CNS neurons that retain the ability to grow following injury, are unable to extend processes beyond the injury induced glial scar due to the presence of inhibitory proteins such as the chondroitin sulphate proteoglycans (CSPGs) aggrecan, versican and neurocan (23;24). These proteins, which inhibit axon guidance (25), have been identified in the human retina and appear during normal development (26). Their response to retinal injury has not been widely investigated, but deposition of CSPGs has been shown to inhibit regeneration of the injured rat optic nerve (27). Degradation of CSPGs using enzymatic digestion by chondroitinase enhances neurite outgrowth and axon regeneration in injured brain (28) and spinal cord (29-31). The use of chondroitinases to degrade CSPGs for treating CNS damage is disclosed in International Patent Application WO 03/074080. The inventors have hypothesised that it is possible that similar treatments may facilitate functional integration of stem cells transplanted into a tissue to be treated, e.g., the retina. Based on the above evidence, the inventors investigated whether CSPG deposition and macrophage/microglia accumulation may prevent the successful migration and integration of stem cells when transplanted into a tissue to be treated.

The difficulties of limited stem cell migration and integration occurs in numerous stem cell therapies. Accordingly, there is a need for a method of improving stem cell migration and integration to thereby improve stem cell therapy.

The present invention provides a composition for improving stem cell integration and/or migration comprising:
a chondroitin sulphate proteoglycan (CSPG) inhibitory agent; and
an anti-inflammatory agent.

The inventors determined that the combination of a CSPG inhibitory agent and an anti-inflammatory agent facilitated integration and/or migration of stem cells into a tissue. In particular, the combination of both agents has been found to be significantly more effective than the use of each individual agent alone.

The composition is for improving stem cell integration and/or migration during any stem cell therapy of a solid organ or solid tissue where it is necessary for the stem cells to integrate and/or migrate in the organ or tissue being treated. The composition can be used for improving stem cell integration and/or migration during stem cell therapy of CNS disorders, visual disorders, heart disease, liver disease, lung disease, kidney disease and muscular disorders. Stem cell therapy of CNS disorders includes any disorder where the CNS tissue needs to be repaired, and include any degenerative disease affecting the CNS such as motor neuron diseases. Suitable disorders of the CNS are described in International PCT application WO 03/074080. Preferred disorders of the CNS that are treatable using stem cell therapy are Parkinson's disease and spinal cord injury. Stem cell therapy of visual disorders includes any disorder where tissue involved in visual function needs to be repaired. Suitable visual disorders that can be treated by stem cell therapy are described in International PCT application WO 2005/054447. Preferred visual disorders that are treatable using stem cell therapy include age-related macular degeneration, macular hole, proliferative diabetic retinopathy, proliferative vitreoretinopathy, retinopathy of prematurity, retinitis pigmentosa and general retinal dystrophies. Stem cell therapy of heart disease includes any disease where heart tissue needs to be repaired. For example, coronary heart disease, cardiomyopathy and congestive heart failure. Stem cell therapy of liver disease includes any disease where liver tissue needs to be repaired. For example, cirrhosis, fatty hepatosis and hepatitis. Stem cell therapy of lung disease includes any disease where lung tissue needs to be repaired, including cystic fibrosis. Stem cell therapy of kidney disease includes any disease where kidney tissue needs to be repaired. For example, renal failure. Stem cell therapy of muscular disorders includes any disorder where muscle tissue needs to be repaired. For example, muscle wasting diseases such as muscular dystrophy.

The term “integration” refers to the ability of the stem cells to functionally integrate within the tissue to be treated so that the stem cells differentiate into the desired cell type and form part of the tissue to be treated. The term “migration” refers to the ability of the stem cells to migrate within the tissue to be treated thereby ensuring that the stem cells are distributed throughout the tissue to be treated. Preferably, the composition of the present invention improves stem cell integration and migration within a tissue being treated.

The composition of the present invention is for use with any appropriate stem cell, including totipotent, pluripotent, multipotent and unipotent stem cells. Preferably, the stem cells are pluripotent, multipotent or unipotent stem cells. The particular type of stem cell to be used with the composition will depend on the stem cell therapy being performed. For example, when the composition is for use in treating a visual disorder, the stem cells are preferably Müller stem cells (see International PCT application WO 2005/054447).

The term “chondroitin sulphate proteoglycan (CSPG)” refers to any CSPG, including NG2, versican, neurocan, brevican, phospacan, aggrecan and hyaluronan, that prevents integration and/or migration of stem cells.

The CSPG inhibitory agent is any agent that reduces or prevents stem cell integration and/or migration inhibitory properties of CSPG. The CSPG inhibitory agent may be an agent that interacts with one or more CSPG to inhibit their inhibitory properties, or eliminates (partially or completely) one or more CSPGs, or reduces the production of one or more CSPGs. Such agents are described in International PCT application WO 03/74080.

Preferably, the CSPG inhibitory agent is an agent that degrades CSPG, for example a chondroitinase, a hyaluronidase or a matrix metalloproteinase, e.g., active MMP2. CSPG inhibitory agents that degrade CSPG are clearly described in International PCT application WO 03/74080. Depending on the CSPG inhibitory agent used, it may be desirable for the composition of the present invention to comprise more than one CSPG inhibitory agent. It is particularly preferred that the CSPG inhibitory agent is chondroitinase ABC (ChABC). The CSPG inhibitory agent is preferably given locally. Methods and formulations for locally delivering active agents are well known to those skilled in the art.

The anti-inflammatory agent can be any agent that reduces or prevents an inflammatory response that reduces or prevents stem cell integration and/or migration. It is particularly preferred that the anti-inflammatory agent prevents the accumulation, activation or production of macrophages or microglia at the desired site of stem cell integration and/or migration. Microglia occur within the CNS and visual system, whereas macrophages occur elsewhere within the body. Accordingly, when the stem cell therapy is of the CNS or the visual system, the anti-inflammatory agent preferably prevents the accumulation, activation or production of microglia. When the stem cell therapy is of any other part of the body, the anti-inflammatory agent preferably prevents the accumulation, activation or production of macrophages. Suitable anti-inflammatory agents include one or more broad acting anti-inflammatory agents. The anti-inflammatory agent may be a steroidal anti-inflammatory agent such as a corticosteroid, e.g., prednisolone, prednisone, fluocinolone, dexamethasone and triamcinolone; a non-steroidal anti-inflammatory agent such as aspirin, salsalate, diflunisal, ibuprofen, ketoprofen, nabumetone, piroxicam, naproxen, diclofenac, indomethacin, sulindac, tolmetin, etodolac, ketorolac, oxaprozin, celecoxib, micocyclin and sulforaphane; or an immunosupressive such as cyclosporine and azathioprine. Other suitable anti-inflammatory agents include antibodies directed against microglia or macrophages. For example, an antibody directed against the CD200 receptor present on microglia may be used to reduce the inflammatory response. Cytokine inhibitors may also be used as anti-inflammatory agents, e.g., anti-TNFα and anti-IL-6. Alternatively, cytokines with immunoregulatory functions such as TGF β1 and IL-10 may be used.

The term “antibody” as used herein refers to polyclonal or monoclonal antibodies of any isotype, or antigen binding fragments thereof, such as Fv, Fab, F(ab′)₂ fragments and single chain Fv fragments. The antibody molecule may be a recombinant antibody molecule, such as a chimeric antibody molecule, a CDR grafted antibody molecule or an antigen binding fragment thereof. Such antibodies and methods for their production are well known in the art.

Depending on the anti-inflammatory agents used, it may be desirable for the composition of the present invention to comprise more than one anti-inflammatory agent. In a particularly preferred embodiment, the composition comprises prednisolone, indomethacine and azathioprine. The one or more anti-inflammatory agents are preferably given systemically or locally. Methods and formulations for systemically or locally delivering anti-inflammatory agents are well known to those skilled in the art.

The composition of the present invention may also comprise stem cells for use in stem cell therapy. The type of stem cells for inclusion in the composition will depend on the therapy as discussed above. Preferably the stem cells are Müller stem cells.

The composition of the present invention may also comprise one or more additional agents that assist with stem cell therapy. Such additional agents include fibroblast growth factor-2 (FGF2) and retinoic acid, and in addition, when the stem cell therapy is for treating visual disorders or disorders of the CNS, neural chemotactic agents, e.g., netrins.

Each agent of the composition of the present invention can be delivered simultaneously, sequentially or separately to the animal undergoing stem cell therapy. The composition can be given repeatedly. Preferably, the anti-inflammatory agent is administered for a sustained period of time to allow any stem cells to integrate. The stem cells and the CSPG inhibitory agent are preferably administered once the anti-inflammatory agent has reduced the inflammatory response in the animal.

The composition is for use in stem cell therapy in any suitable animal, such as a human, livestock or pets. Preferably the animal is a mammal or a bird. In particular, the animal may be selected from the group comprising: human, dog, cat, cow, horse, pig, sheep and birds. It is specifically preferred that the animal is a human.

The present invention also provides a chondroitin sulphate proteoglycan (CSPG) inhibitory agent and an anti-inflammatory agent for improving stem cell migration and/or integration in stem cell therapy.

The present invention also provides a method for improving stem cell migration and/or integration in stem cell therapy of an individual comprising delivering an effective amount of a chondroitin sulphate proteoglycan (CSPG) inhibitory agent and an anti-inflammatory agent to the individual.

The present invention also provides the use of a chondroitin sulphate proteoglycan (CSPG) inhibitory agent and an anti-inflammatory agent in the manufacture of a medicament for improving stem cell migration and/or integration in stem cell therapy.

Each agent can be delivered simultaneously, sequentially or separately to the individual undergoing stem cell therapy. The stem cells for use in the stem cell therapy may be administered with the agents simultaneously, sequentially or separately.

The present invention also provides a pharmaceutically acceptable composition comprising a chondroitin sulphate proteoglycan (CSPG) inhibitory agent and an anti-inflammatory agent, together with one or more pharmaceutically acceptable excipients.

The pharmaceutically acceptable composition may additionally comprise stem cells for use in stem cell therapy.

Suitable excipients are well known to those skilled in the art.

The specific amounts of each agent can be determined using standard methodologies and by extrapolating from the specific values used in the example section below. The specific amounts used will depend on a number of factors, including the size and metabolism of the animal to be treated.

The pharmaceutical compositions of the present invention may be administered in any suitable manner, including orally, parenterally or via an implanted reservoir. Preferably the pharmaceutical composition is administered by injection. More specifically, it is preferred that the anti-inflammatory agent is delivered systemically or locally, and the CSPG inhibitory agent is delivered locally.

The pharmaceutical composition may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent, dispersant or a similar alcohol as described in Ph. Helv.

The pharmaceutical composition of this invention may be orally administered in any orally acceptable dosage form including, but not limited to, capsules, tablets, and aqueous suspensions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are administered orally, the active ingredient is combined with emulsifying and suspending agents. If desired, certain sweetening and/or flavouring and/or colouring agents may be added.

The present invention also provides the pharmaceutical composition of the present invention for use in therapy, especially stem cell therapy.

The present invention also provides a kit for improving stem cell migration and/or integration during stem cell therapy comprising:
a chondroitin sulphate proteoglycan (CSPG) inhibitory agent; and
an anti-inflammatory agent.

The kit may additionally comprise stem cell for use in the stem cell therapy.

The present invention is now described by way of example only with reference to the following figures.

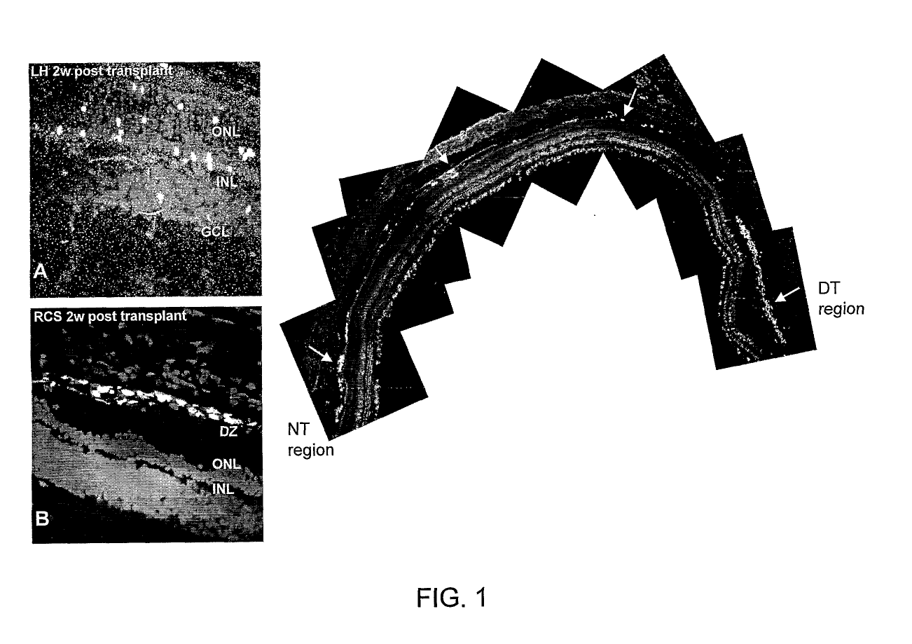

FIG. 1 shows the migration of Müller stem cells following subretinal transplantation into the neonate LH and dystrophic RCS rats. (A) Migration of Müller stem cells (GFP labelled) into the inner and outer nuclear cell layers (INL and ONL) 2 weeks after transplantation into a 2 day old LH rat. (B) Müller stem cells (GFP labelled) remained in the subretinal space 2 weeks after transplantation into a 5 week old RCS rat. (C) Montage of whole retina to show widespread Müller stem cell migration along the subretinal space 5 weeks after subretinal injection into a 5 week old RCS rat.

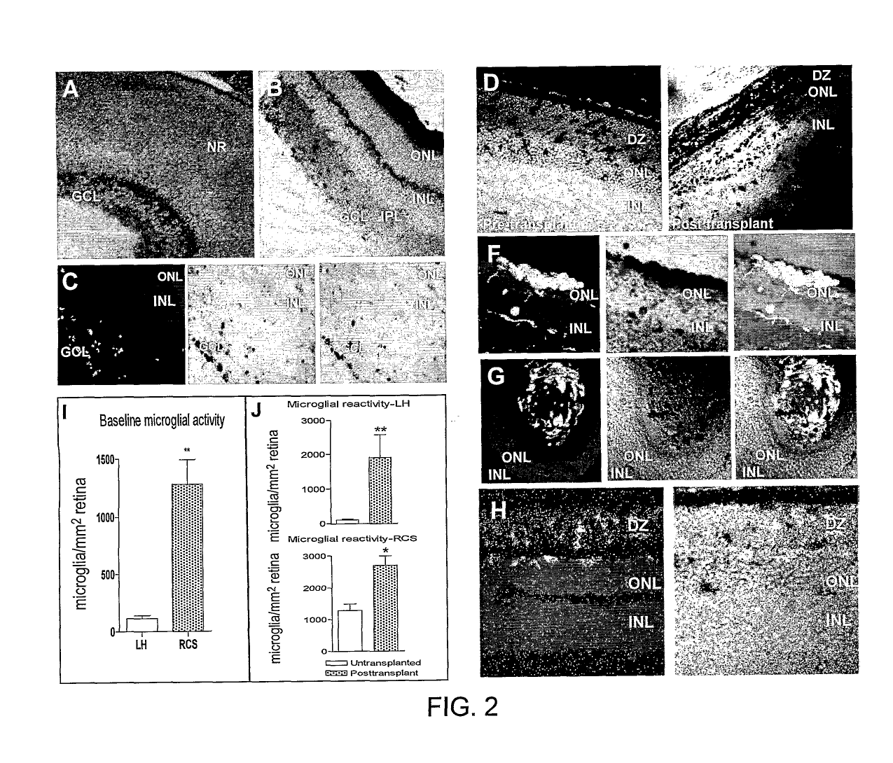

FIG. 2 shows the distribution of microglia in the LH and RCS rat retina before and after Müller cell transplantation. (A) Section of neonate LH rat retina (3-day old animal) showing minimal CD68 reactivity (black cells) in the ganglion cell layer (GCL). (B) Section of LH rat retina 2 weeks after transplantation, showing microglia accumulation in the GCL and inner plexiform layer (IPL) (black cells). (C) Section of LH rat retina 2 weeks after transplantation into a two day old animal. Left figure shows migration of grafted Müller cells (GFP labelled) into the GCL. Middle figure shows Nomarski illumination of the same retinal section, demonstrating accumulation of cells expressing CD68 (black cells) in the GCL. Right figure illustrates Nomarski illumination of the same section, showing association of microglia (black cells) with transplanted Müller stem cells (GFP labelled). (D) Nomarski illumination of a non transplanted RCS rat retina at 5 weeks of age showing severe infiltration of microglia (black cells) in the outer segment debris zone (DZ) and the outer nuclear layer (ONL). (E) Nomarski illumination of RCS rat retinal section 2 weeks after transplantation in a 5 week old animal. Microglia (black cells) can be observed thorough all retinal cell layers. (F) Left figure shows accumulation of transplanted cells in the subretinal space. Middle image shows Nomarski illumination identifying the localization of CD68 positive cells (black staining) in the same retinal section. Right figure shows Nomarski illumination identifying co-localization of transplanted cells (GFP labelled) with microglial cells expressing CD68 (black staining). (G) Left figure shows confocal image of retinal section of RCS rat transplanted at 5 weeks of age. Transplanted cells can be seen forming a large cluster in the subretinal space 2 weeks after transplantation. Middle image shows Normaski illumination of the same section to indicate localization of microglia around the transplanted cells (black cells). Right figure shows microglia (black staining) surrounding the transplanted cells and resembling a granuloma-type structure. (H) Retinal section of a RCS rat treated with oral prednisolone and intraperitoneal indomethacin along with cyclosporine and azathioprine. Confocal image (left) shows limited migration of grafted cells into the debris zone (DZ), and reduced infiltration of microglia in the same region as observed under Nomarski illumination on the right. (I) Histogram shows that there is a significantly higher number of microglia in a 5 week old RCS retina compared to a neonatal LH retina (p=0.0055). (J) Upon transplantation there is a significant increase in microglial accumulation. ** p<0.001 LH transplanted compared to non-transplanted retina; *p<0.05 RCS compared to non-transplanted.

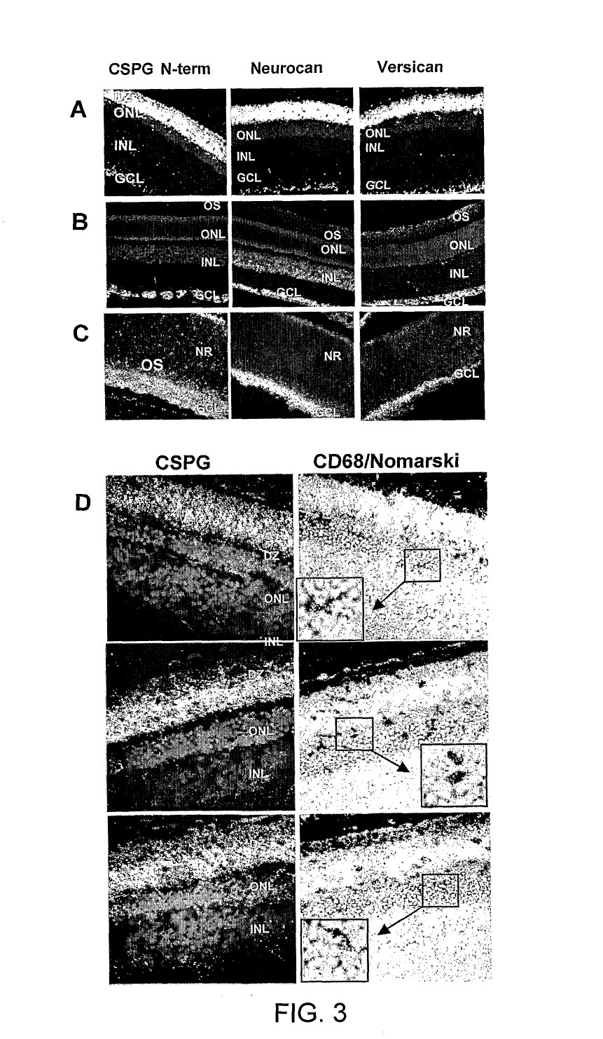

FIG. 3 shows the expression of CSPGs in the retina of non-transplanted normal LH and dystrophic RCS rats and CSPG co-localization with microglia. (A) Confocal images showing accumulation of the N-terminal region of CSPGs, neurocan and versican in the debri zone (DZ) of retinal sections from a non-transplanted 5 week old RCS rat. (B) Retinal sections of a non-operated LH rat at 5 weeks of age lacking expression of CSPGs in the outer nuclear layer (ONL) but staining for these molecules in the ganglion cell layer (GCL). (C) Retinal sections of non transplanted neonate LH rat retina showing expression of CSPGs in the inner plexiform layer and developing GCL. (D) Confocal retinal images of non-transplanted 5 week old RCS rats showing accumulation of the N-terminal of CSPGs, neurocan and versican on the left, and co-localization of these proteins with cells expressing CD68 (black staining) as observed under Nomarski illumination on the right. Cells surrounded by squares are magnified in the insets at the bottom of each figure to show details of microglial co-localization with CSPGs.

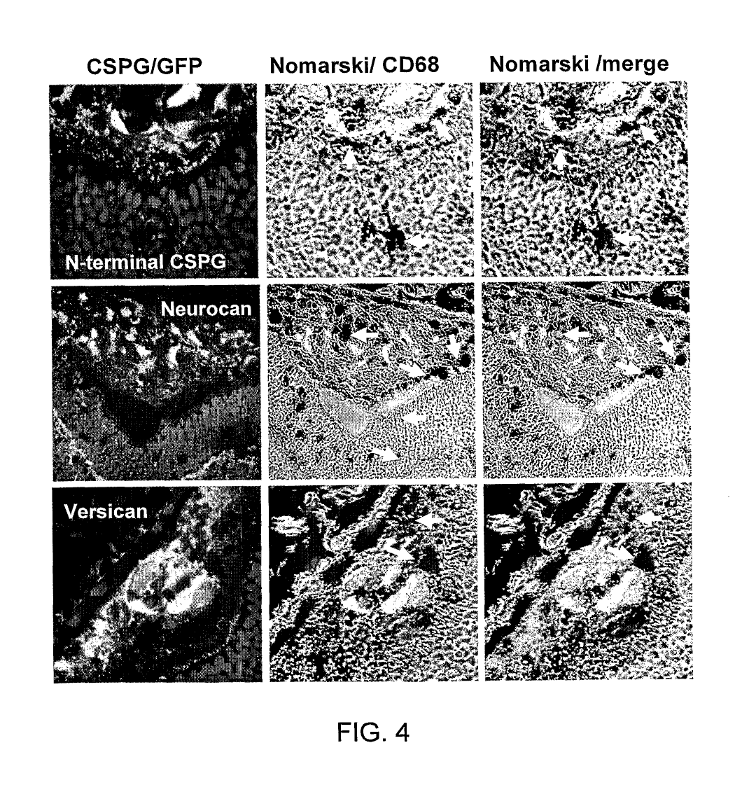

FIG. 4 shows the inhibition of Müller stem cell migration by chondroitin sulphate proteoglycans. Confocal images of retinal sections from 7 week old RCS rats 2 weeks after subretinal transplantation of Müller stem cells. Sections on the left column shows the transplanted cells (GFP labelled) surrounded by N-terminal CSPG, neurocan and versican. The middle column shows the same sections under Nomarski illumination to illustrate the accumulation of CD68 reactive cells (black). The column on the right shows the merged images under Nomarski illumination illustrating co-localization of CD68 positive cells and CSPGs surrounding the transplanted cells (GFP labelled). Nuclei are stained with DAPI.

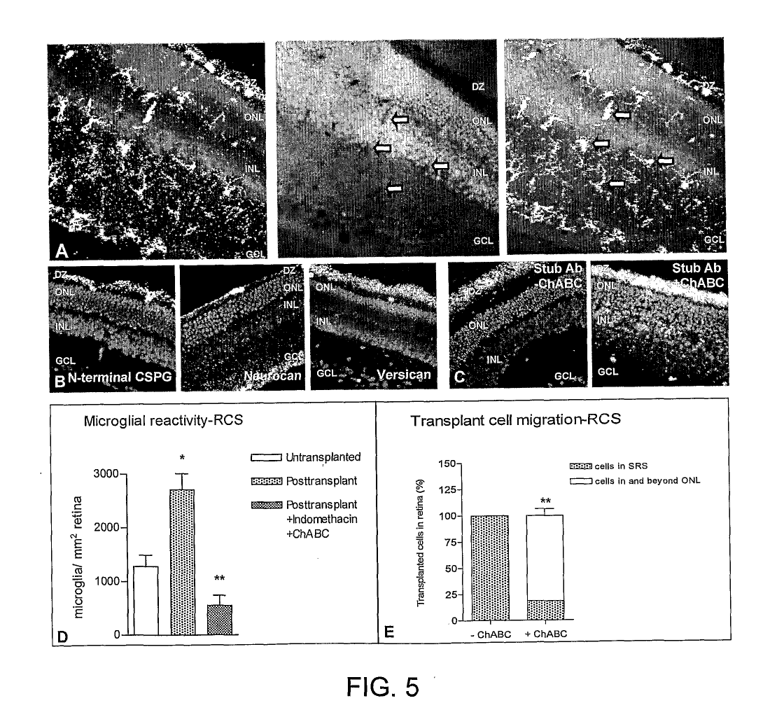

FIG. 5 shows the digestion of CSPGs and enhanced microglial suppression facilitates migration of grafted Müller stem cells. (A) Micrograph on the left shows a confocal image of a retinal section from a 7 week old RCS rat following 2 weeks after Müller stem cell transplantation in the presence of ChABC. Animals were transplanted at 5 weeks of age and treated with enhanced immune suppression for the duration of the experiment. Transplanted cells (GFP labelled) can be observed throughout the whole width of the retina. Middle image shows the same retinal section under Nomarski illumination to illustrate retinal infiltration by CD68 positive cells (black) showed by arrows. Right image shows the same section under Nomarski illumination to illustrate the colocalization of CD68 positive cells with the transplanted cells (GFP labelled) that had migrated into the retina (arrows). (B) Confocal images from retinal sections of a 7 week old RCS rat 2 weeks after injection of ChABC and enhanced immune suppression. Sections stained for CSPGs showed a marked reduction in the expression of the N-terminal region of CSPGs, neurocan and versican. (C) Image on the left shows a retinal section of a 5 week old non transplanted RCS rat staining for the stub epitope. Spontaneous degradation of CSPGs is shown by the staining. Image on the right shows a retinal section of a 7 week old rat injected in the subretinal space at 5 weeks of age with ChABC. Staining indicates the exposure of the stub epitope upon degradation of CSPGs by this matrix degrading enzyme. (D) Histogram shows that Müller stem cell transplantation into the subretinal space of RCS rats induces a marked increase in the number of infiltrating microglia (*p<0.05 compared to non-transplanted animals) and that enhanced immune suppression by addition of oral prednisolone and intraperitoneal indomethacine to the standard regimen, induced a significant reduction in the number of infiltrating retinal microglia (**p<0.001 compared to transplanted animals). (E) Histogram depicting increased migration of Müller stem cells into the inner retinal layers when transplanted with ChABC. No cells were detected beyond the ONL in the control animals (−ChABC) while nearly 80% of cells migrated beyond the INL into the inner retinal layers with ChABC (**p=0.0011).

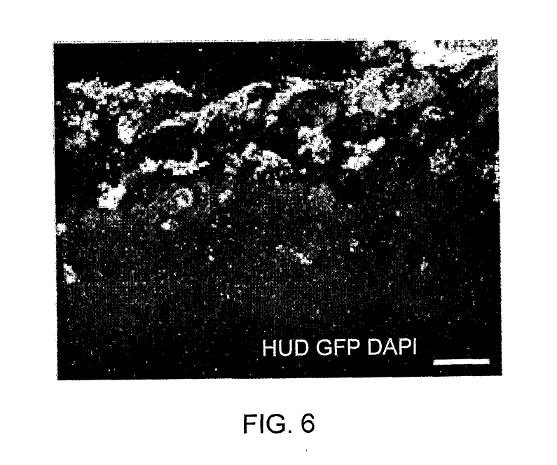

FIG. 6 shows a confocal photo-micrograph of a Lister hooded rat retina following depletion of RGC by NMDA and injection of Müller stem cells differentiated into cells expressing RGC markers: This image shows a retinal section following 3 weeks after transplantation with cell preparations of Müller stem cells differentiated into cells expressing RGC markers (GFP fluorescent cells). These cells were shown to have migrated into the RGC layer. Section counterstained with DAPI (blue). GCL, ganglion cell layer; IPL, inner plexiform layer; INL, inner nuclear layer; ONL, outer nuclear layer.

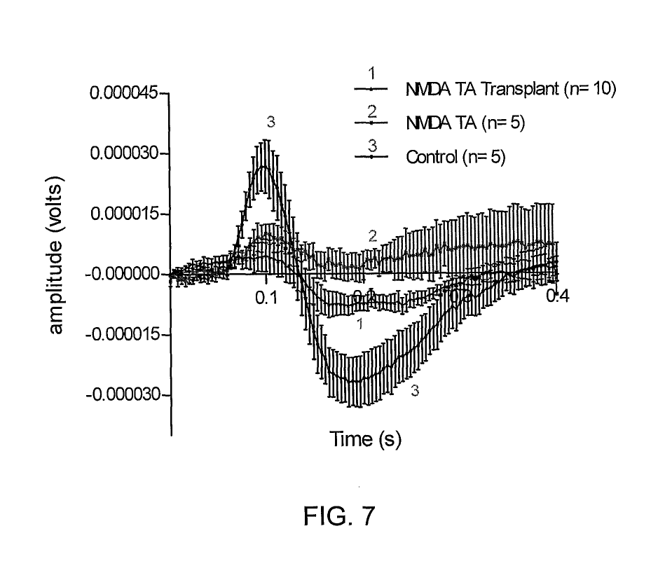

FIG. 7 shows the Scotopic Threshold Response (STR) of NMDA-Triamcinolone (TA) treated eyes from Lister hooded rats 2 weeks post-transplantation of an enriched population of cells expressing RGC markers. Control eyes (3) show a characteristic STR curve with a positive response (pSTR) followed by a larger negative response (nSTR). NMDA TA treated eyes only show a small pSTR) (2). The NMDA TA eyes which have been transplanted with differentiated Müller stem cells have a small pSTR and also demonstrate a partial recovery of the nSTR (1) which is significantly increased when compared with that of eyes treated with NMDA TA only.

### EXAMPLES

### Materials and Methods

### Animals and Immunosuppression

Dystrophic RCS rats and adult and neonatal Lister hooded rats were used in the study. All rats were maintained according to the Home Office regulations for the care and use of laboratory animals and the UK Animals (Scientific Procedures) Act (1986). Dystrophic RCS rats were bred in-house, kept under 12 h/12 h light-dark cycle (light cycle mean illumination: 30 cd/sqm.) and transplanted at the age of 5-6 weeks (35-40 days) by which time the retinal degeneration is well established. Lister hooded rats were purchased from Harlan (UK). Animals were normally immunosuppressed with oral Cyclosporine A (Sandimmun, Sandoz, Camberley, UK, 210 mg/litre of drinking water,) and Azathioprine (Sigma, UK; 20 mg/litre) from 2 days before transplantation until termination of the experiment. When using additional immunosuppression, animals received oral prednisolone (Sovereign Medical, UK, 5 mg/liter) in addition to Cyclosporine A and Azathioprine, together with daily intraperitoneal injections of indomethacin (0.1 mg/100 gm body weight) for the duration of the experiment. For transplantation into neonatal Lister hooded pups, pregnant dams were immunosuppressed with oral Cyclosporine A and Azathioprine as above.

Isolation and Preparation of Müller Cells with Stem Cell Characteristics

Isolation of Müller stem cells from the neural retina of donor human eyes consented for research was performed as previously described (32). Briefly, neural retina sectioned at least 2 mm away from the ora serrata was incubated in trypsin-EDTA (0.5% trypsin/0.2% EDTA; Invitrogen, Paisley, Scotland) for 20 mins at 37° C. After vigorous trituration, released cells were washed and suspended in DMEM containing L-L-glutamax 1 (Invitrogen, Paisley, Scotland), 10% foetal calf serum (FCS, Invitrogen, Paisley, Scotland) and 40 ng/ml epidermal growth factor (EGF, Sigma, UK). Cells were plated onto fibronectin coated tissue culture dishes and cultured for 2-3 weeks until formation of adherent cell colonies. Colonies were detached and transferred onto new culture dishes and culture continued in the above medium without EGF. Upon confluence, cells were examined for their stem cell characteristics as previously described (18). Cells that underwent more than 50 passages without loosing their stem cell characteristics were used for transplantation.

### Preparation of Cells for Transplantation

Müller stem cells used for transplantation were transfected with an immunodeficiency virus type 1 (HIV-1) based lentiviral vector expressing low toxicity hrGFP from a spleen focus forming virus (SFFV) promoter, previously described as 1-schrgfpw (33). Confluent cells plated in a 12 well culture dish were infected with 1-schrgfpw at a multiplicity of infection of one transducing unit per cell in the presence of polybrene (10 ug/ml, Chemicon, USA). Using this method over 80% of Müller stem cells expressed GFP following 1 week after infection. The transfected cells were grown to confluence in a 25 cm² flask and green fluorescent cells were selected by FACS cell sorting using a BD FACScalibur, UK. To ensure that the lentiviral vector did not modify the stem cell characteristics of the transfected cells, these were examined for the expression of stem cell markers and sphere formation as previously described (18). Three days prior to transplantation, lentivirus-GFP transfected cells were plated onto a 75 cm² flask and allowed to reach about 70% confluence. On the day of the transplant, cells were trypsinised, counted and resuspended in serum free medium to a concentration of 2×10⁴ cells/μl.

### Sub-Retinal Transplantation

Rats were anaesthetised with an intraperitoneal injection of Ketamine HCl (Ketaset, Fort Dodge Animal Health, Southampton, UK) and Medetomidine HCl (Domitor, Pfizer, Sandwich, UK). The pupils were dilated using 1% Tropicamide and 2.5% Phenylephrine (Chauvin Pharmaceuticals, UK) before injection of 2 μl of the cell suspension into the subretinal space of adult dystrophic RCS rats (n=9) using a 30G metal needle attached to a Hamilton syringe, under direct visualisation with a Leitz operating microscope. Sham injected rats received DMEM medium without cells (n=6). Neonatal Lister hooded rats (postnatal day 2-PN2), were anaesthetised with 1/10^{th} the adult dose of Ketamine and Medetomidine by intraperitoneal injection. The lids were opened surgically and 1 μl of cell suspension (n=11) or medium (n=9) was injected into the subretinal space using a 32G metal needle attached to a 2.50 Hamilton syringe. When treating RCS retinae with Chondroitinase ABC (n=8), cells were suspended at a concentration of 4×10⁴ cells/μl. Prior to transplantation, 1 μl of the cell suspension was mixed with 1 (0.01 U) of Chondroitinase ABC (ChABC) (Seikagaku Corporation, Tokyo, Japan) and the mixture injected into the sub retinal space as indicated above.

### Tissue Processing and Immunohistochemistry

Two weeks post-transplantation, rats were terminally anaesthetised with intraperitoneal sodium pentobarbitone and perfused transcardially with phosphate buffered saline (PBS) followed by 4% paraformaldehyde (PFA) in 0.1M phosphate buffer. Eyes were excised, post-fixed in PFA for one hour, cryoprotected with 30% sucrose overnight and embedded in optimal cutting temperature compound (OCT, VWR, Lutterworth, UK). Cryostat sections 14 μm thickness were placed onto precharged slides and the slides air dried prior to storage at −80° C. Sections with visible transplants (in eyes which underwent cellular transplantation) (untreated RCS n=4, neonatal Lister hooded ChABC treated RCS n=5) and sections with visible transplant sites (in sham operated eyes) (RCS n=3, Lister hooded n=3) were selected for immunostaining. Sections from 2 different un-operated eyes in each group were used as controls.

Dried slides were blocked with 5% donkey serum in PBS/0.3% triton and reacted with primary antibodies diluted in the same blocking solution overnight at room temperature (RT). The primary antibodies used were CSPG (CS56, Sigma, monoclonal, 1:200), neurocan (IF6, Developmental studies hybridoma bank (DSHB), University of Iowa, USA, monoclonal, 1:100), versican (12C5, DSHB, monoclonal, 1:100), ED1 (anti rat-CD68, Serotec, monoclonal, 1:1000)—a marker for rat macrophage/microglia, and 2B6 (Seikagaku, Japan), an antibody that recognizes an epitope exposed following chondroitinase ABC degradation of chondroitin-4 sulfate. After incubation with primary antibodies overnight at room temperature (RT), sections were washed in PBS and incubated with the relevant Alexa secondary antibodies (mouse, rabbit or goat 488 or 555, raised in donkey, Invitrogen, Paisley, Scotland) diluted 1:500 in PBS plus 2% donkey serum for 1.5 h at RT. After washing, sections were counterstained with 4′,6-diamidino-2-phenylindole (DAPI, Sigma, UK) washed with Tris buffer (0.05M, pH 7.4) and mounted in Vectashield (Vector Laboratories Ltd, Peterborough, UK). Sections were also processed in parallel with secondary antibodies alone which served as negative controls.
Macrophage/microglial infiltration was determined by co-staining of retinal sections with a mouse monoclonal antibody to CD68 antigen (EDI, Serotec, UK). Antibody reactivity was determined by visualization with 3-3′ diaminobenzidine (DAB; Sigma, UK) enhanced with aqueous 1% nickel ammonium sulphate and aqueous 1% cobalt chloride by a modification of published methods (34) as follows. Sections were blocked and incubated with the anti CD68 antibody overnight at RT, using the same blocking solution used with other antibodies in the study (see above), with the addition of 0.5% bovine serum albumin (BSA; Sigma, UK). After washing, sections were incubated in biotinylated horse anti-mouse secondary antibody, rat adsorbed (1:150, Vector Laboratories, Peterborough, UK) followed by streptavidin-horseradish peroxidase (Vector Laboratories, Peterborough, UK). They were then developed with a 25 s incubation in DAB solution (0.05% in 0.1M PBS with 1% nickel ammonium sulphate and 1% cobalt chloride in hydrogen peroxide) and cells expressing CD68 were detected by their characteristic brown-black staining under Nomarski illumination.
To identify other markers in sections stained for CD68, slides were then washed, blocked and incubated with other primary antibodies (see above) and detected with fluorescence-labelled antibodies. Sections were photographed using a Zeiss LSM 510 confocal microscope and images were analysed using the Zeiss LSM Image Browser software (Carl Zeiss, Oberkochen, Germany).

### Results

Migration and integration of Müller stem cells following subretinal grafts into neonate LH and RCS rats.

Two weeks after transplantation of Müller stem cells into the subretinal space of neonate (2 days old) LH rats, migration of cells into the inner retinal cell layers was often observed. However, relatively few cells migrated and this migration only occurred into the area adjacent to the transplantation site (FIG. 1A). In contrast, two weeks after transplantation of these cells into the subretinal space of 4-5 week old RCS rats, (the age at which the photoreceptor cell layer has been reduced to about half of its normal thickness) cells accumulated at the interface between the outer segment debris zone (DZ) and the retinal pigment epithelium (RPE) and often gave the appearance of small cell aggregates (FIG. 1B). Cells rarely migrated into the inner retina. Examination of the whole RCS retina 2 weeks after transplantation showed that cells predominantly migrated along the subretinal space lining the DZ (FIG. 1C). It was of interest that cells that had been grafted in the subretinal space of the dorso temporal region were often seen along the entire subretinal space, but not within the retina itself (FIG. 1C).

Microglia Response to Retinal Transplantation of Müller Stem Cells

Since microglia are known to migrate and proliferate within the degenerating retina of the RCS rat (20-22), the inventors investigated whether these cells play a role in the inhibition of Müller cell migration following subretinal transplantation into 5 week old RCS rats. The inventors also compared this response with that of neonatal LH rats. Examination of the presence of microglia in LH neonate retina showed only occasional CD-68 positive cells within the ganglion cell layer (GCL) of 2 day old animals (FIG. 2A). However, 3 weeks after transplantation an increase in the number of CD68 positive cells was observed in the GCL and inner plexiform layer (IPL) of LH rats, despite immune suppression with oral cyclosporine and azathioprine (FIG. 2B). Confocal examination of retinal sections from transplanted neonatal LH rats stained for CD68 and GFP showed that although Müller stem cells had migrated into the retina, microglial reactivity was often associated with the grafted cells (FIG. 2C).

Unlike the mild microglia activation observed in the neonate and transplanted LH retina, a marked accumulation of CD68 positive cells was observed in the retina of 5 week old RCS rats. Numerous microglia were observed in the outer segment DZ and in the outer nuclear layer (ONL) (FIG. 2D). Two weeks after Müller stem cell injection into the subretinal space, adult RCS rat retina showed a more severe and widespread migration of microglia (FIG. 2E). This also occurred despite immune suppression with oral cyclosporine and azathioprine, which suggested that microglial activation was not related to lymphocyte invasion. Confocal microscopy of transplanted retinal sections co-stained for CD68 and GFP showed that Müller stem cells that had not migrated into the retina and that lined the outer segment DZ, appeared to have been phagocytosed by microglia, as judged by the co-localization of both CD68 and GFP (FIG. 2F). Moreover, aggregates of grafted Müller stem cells were often observed in the subretinal space of the transplanted RCS rat surrounded by microglia (FIG. 2G). These CD68 positive cells appeared to be halting the migration of Müller stem cells into the retina (FIG. 2G). In order to promote cell migration by diminishing microglial activation, animals were given oral prednisolone and daily intraperitoneal injections of indomethacin, in addition to oral cyclosporine and azathioprine, for the duration of the experiment. As observed in FIG. 2H, although microglial numbers appeared comparatively reduced with additional prednisolone and indomethacin treatment, transplanted cells continued to be associated with microglia and remained in the DZ unable to migrate into the retina. Quantitative comparison of the infiltrating microglia in the LH and RCS rat prior to and following transplantation showed that the degenerating RCS rat retina harboured significantly higher numbers of microglia than the normal LH rat retina (p<0.01) (FIG. 2I), and that Müller stem cell transplantation caused a marked increase in the number of microglia in both the LH (p<0.001) and RCS (p<0.05) rat retina (FIG. 2J).
Expression of CSPGs in the Normal and Degenerated Rat Retina and Association of These Proteins with Microglia

Immunostaining of the unoperated 5 week old RCS rat retina for the CSPG N-terminal region (common to all CSPGs) revealed marked expression of this molecule in the outer segment DZ (FIG. 3A). Strong staining for neurocan and versican was also observed in the same region in unoperated (FIG. 3A) and sham-operated animals (not shown). This contrasted with the expression of these proteins observed in 4 week old LH rats, where staining for these molecules was minimal or absent in the OS region of the retina (FIG. 3B). In the adult LH retina staining for the N-terminal of CSPGs, neurocan and versican was seen in the ganglion cell layer (FIG. 3B). Expression of these proteins was also detected in the inner plexiform layer and in the ganglion cell region of the 2 day old LH rat retina (FIG. 3C).

Co-staining for CSPGs and CD68 in retinal sections from 5 week old RCS rats showed that microglia were mainly localized at the sites where accumulation of CSPGs could be observed (FIG. 3D). Furthermore microglia observed outside the DZ and infiltrating the ONL co-stained for the N-terminal of CSPGs as well as neurocan and versican, suggesting that microglia may constitute a source of CSPGs in the degenerating RCS rat retina (FIG. 3D).
Inhibition of Müller Stem Cell Migration by CSPGs

To elucidate the role of CSPGs on the inhibition of Müller stem cell migration and integration 3 weeks after transplantation into the subretinal space of 5 week old RCS rats, the inventors examined retinal sections of grafted animals for the expression of CSPGs and CD68. Confocal microscopic analysis of grafted RCS rat retinae showed that Müller stem cells that failed to migrate into the retina were often surrounded by the N-terminal of CSPGs, neurocan and versican (FIG. 4). CSPGs often formed a pericellular cuffing with severe microglial activation. Colocalization of microglia with all the CSPGs investigated was observed in all specimens examined (FIG. 4). This spatial correlation of ECM proteins and CD68 expression around the transplanted cells suggest that in addition to CSPGs accumulating in the degenerating retina, CSPGs released by activated microglia are also likely to contribute to the inhibition of cell migration and integration.

Effect of Combined CSPG Digestion and Microglial Suppression on the Migration of Grafted Müller Stem Cells

To further characterise the contribution of CSPG to inhibition of graft cell migration and integration, the inventors transplanted 5 week old RCS rats with Müller stem cells together with ChABC to promote matrix degradation and promote cell migration. The inventors also used enhanced microglial suppression on these animals, combining oral cyclosporine A, azathioprine and prednisolone with daily intraperitoneal injections of indomethacin for the duration of the experiment. Two weeks post transplantation the inventors saw a dramatic improvement in the migration of grafted cells through the entire thickness of the retina in eyes treated with ChABC (FIG. 5A). Many of the migrating cells interestingly bore characteristic neuronal morphology (FIG. 5A). The inventors quantified this change in migration by counting the total number of grafted cells detected in the retina and classifying them based on their localisation to the subretinal space (SRS) or to the inner retinal layers beyond the ONL. Almost 80% of the cells were found to have migrated into the inner retinal layers when transplanted in conjunction with ChABC. This was in contrast to the control animals where almost all of the cells remained in the subretinal space (t test, n=4, p=0.0011).

Reduction in CSPG expression in eyes treated with ChABC was confirmed by a decreased staining for CSPG N-terminal, neurocan and versican in the outer segment DZ (FIG. 5B) when compared with untreated retinas (FIG. 3D). In addition, to confirm that reduction in CSPG expression was indeed a result of ChABC digestion, the inventors stained untreated and ChABC treated retinal sections with the chondroitin sulphate stub (CS stub) IB5 antibody which detects CS stub epitopes exposed on CSPG molecules by ChABC enzymatic digestion. As seen in FIG. 5C (+ChABC), the ChABC treated retinae showed widespread staining for the stub antibody throughout the whole retinal thickness, indicating that CSPGs were reduced as a result of ChABC digestion. Interestingly, untreated retinae also showed a localized staining for the CS stub epitopes in the outer segment DZ (FIG. 5C −ChABC), suggesting that some degree of CSPG digestion also occurs during photoreceptor degeneration. Despite the ChABC enzymatic activity, the retinal architecture of treated animals appeared to be well preserved (FIG. 5A).

With the use of enhanced microglial suppression in the ChABC treated animals, in addition to improved migration of grafted cells, there is a decrease in the amount of microglial accumulation (FIG. 5A). Quantitative analysis showed that the number of microglia within the retina of animals treated with ChABC and enhanced immune suppression, was significantly lower than in animals transplanted without additional immune suppression (Bonferroni p<0.001). (FIG. 5D). Despite the reduction in microglial infiltration, many cells which had migrated and acquired neural morphology were co-localized (though less frequently) with CD68 positive cells (FIG. 5A arrows). These observations suggest that CSPGs and microglia form a considerable physical barrier to successful retinal transplantation.

Successful migration and integration of stem cells to restore tissue function may depend on the appropriate interaction of the host environment with the transplanted cells. Various studies have shown that when stem cells are transplanted to regenerate retina, migration and integration of grafted cells is better in an immature retina or in a retina subject to acute injury (11;27). However, little consideration has been given to the migration and integration of stem cells in long standing retinal degeneration and inflammation. It is important to understand that tissue that needs repair has often undergone a series of inflammatory and degenerative changes, which may limit the functional integration of grafted stem cells. The present observations confirm previous reports that the neonatal retina provides a permissive environment in which stem cells can migrate and integrate. However, our results show that although grafted Müller stem cells migrated into the neonate LH retina, these were often found in association with cells expressing CD68, a marker of macrophage/microglia. Microglial activation may occur as an innate response to the injection injury itself as well as to the grafted cells. Our observation that microglia localized to the sites where grafted cells had migrated strongly suggests that microglia play an important role in preventing the migration and integration of transplanted stem cells. In accordance with previous studies, considerable microglial activation was seen in the degenerating retina of the dystrophic RCS rat prior to transplantation (20-22). This activation was greater than that observed in the neonatal and transplanted adult LH rat retina. It is likely that the extensive baseline microglial activity in the RCS rat retina prior to transplantation mounts a rapid response to cell transplantation thereby preventing the migration of the transplanted cells. Stem cell clusters were often seen in the subretinal space, densely surrounded by microglia, resembling granuloma-like structures. These clusters were not observed in the LH rat after transplantation into the neonate retina, suggesting that in the absence of pre-existing microglial reactivity the grafted cells were able to migrate into the retina. It is possible that the xenograft nature of the transplant (human cells to rat retina) further enhanced the microglia response, but allogeneic transplantation of Müller stem cells in the RCS rat retina resulted in similar microglia reactivity (data not shown). Activated microglia present in the retina prior to transplantation may not only prevent the initial migration of grafted cells into the retina, but may also phagocytose and degrade them.

CSPGs are produced during glial scarring in the central nervous system (24), and their role as inhibitory axon guidance molecules (25;35) is well documented. The inventors investigated the expression of these proteins in the degenerating retina of the dystrophic RCS rat to identify whether they are also inhibitory in the integration of transplanted cells into the retina. The inventors found selective accumulation of CSPGs including neurocan and versican in the DZ of the OS of the degenerating retina. This is in accordance with other studies that show heavy accumulation of chondroitin sulfates (36) and neurocan (37) in the retina of the dystrophic RCS rat upon degeneration. The inventors' results further demonstrated that microglia, which are known to produce CSPGs in vitro (38) and in vivo upon spinal cord injury (39;40), also express the N-terminal fraction of CSPGs, as well as neurocan and versican in the degenerating RCS rat retina. Significantly, co-staining of retinal sections from transplanted RCS rat retinae for CSPGs and CD68 expression, showed that microglia surrounding the grafted cells stained for CSPGs. These observations strongly suggest that one of the mechanisms by which microglia might be inhibiting stem cell migration and integration into the damaged retina is by releasing CSPGs.
Since stem cell migration from the site of transplantation into the damaged or injured retina is crucial to retinal repair, the inventors aimed to facilitate cell migration by inhibiting the microglial response in RCS rats using intra-peritoneal indomethacin and a combination of oral prednisolone, cyclosporine and azathioprine (18). Despite a reduction in microglial accumulation in the vicinity of the transplants as a result of this treatment, residual microglial reactivity was still observed in association with the grafted cells. Microglia remained in the DZ, where CSPGs accumulation was observed in the degenerating retina of RCS rats, indicating that microglial suppression alone is not sufficient to promote migration of the grafted cells.
Degradation of CSPGs by chondroitinase has been shown to promote migration of transplanted cells and regenerating axons through glial scars, particularly in the spinal cord, (29-31) and in the retina it has been used to improve lentiviral vector mediated transduction of photoreceptors (41). On this basis the inventors used this enzyme to break down CSPGs accumulating in the degenerating RCS retina. When Müller stem cells were transplanted with ChABC in conjunction with oral azathioprine/cyclosporine and intraperitoneal indomethacin to control the microglial response, the inventors observed a dramatic improvement in the migration of these cells. The inventors were able to demonstrate efficient breakdown of CSPGs through a marked reduction in the expression of these proteins and appearance of the stub epitope in the transplanted retina following enzymatic digestion by ChABC. The importance of ChABC in promoting transplant cell integration might however extend beyond its effect on cell migration and on to facilitating neurite extension by these cells. This is suggested by the observation that Müller stem cells also adopted a neuronal morphology when transplanted in the presence of this enzyme. Extensive work on the role of CSPG in neuronal plasticity has revealed that the deposition of this ECM protein is also inhibitory to the formation of new neuronal synaptic connections and is a mechanism by which the mature mammalian nervous system protects itself from the formation of aberrant neuronal synapses when injured (42). ChABC has been shown to restore synaptic plasticity by breaking down the CSPG-rich perineural nets in the visual cortex of mature rats (43;44). Most recently this theory has gained further support in the retina with reports of improved synapse formation of transplanted photoreceptor precursors with host neurones when delivered in conjunction with ChABC (45).
From the present observations it can be concluded that an environment of long standing injury, degeneration and inflammation may be inhibitory to integration of transplanted stem cells due to the accumulation of connective matrix, e.g., CSPGs, and an inflammatory response, e.g., the presence of microglia. Although total suppression of microglia was not achieved in the present study, reduction of these scavenger cells led to increased migration of grafted Müller stem cells. Combination of CSPG degradation and microglia suppression led to robust graft cell migration into the retina, although remaining microglia continued to be associated with the transplanted cells. Break down of the physical barrier created by CSPGs can greatly enhance migration of transplanted cells into a diseased retina, but is not sufficient to ensure successful integration. Macrophage/microglia reactivity along with migratory barriers must be overcome in order to achieve integration of stem cells.
Differentiation of Injected Müller Stem Cells into Retinal Ganglion Cells (RGC)

Further results show that a single injection of Müller stem cells differentiated into retinal ganglion cells (RGC) combined with triamcinolone and Chondroitinase ABC facilitates migration and integration of these cells into the RGC layer of Lister hooded rats (see FIG. 6). These animals had been depleted of RGC by previous injection of the neurotoxin NMDA (46). In this case the anti-inflammatory agent was administered locally into the eye. The Triamcinolone formulation used is the same one used in patients—Triamcinolone Acetonide (TA) (Kenalog) 40 mg/ml. However given the toxic nature of the carrier solution, prior to the injection, we centrifuged the TA solution and removed the carrier containing supernatant. The TA particles were then resuspended and washed thoroughly in 2 changes of sterile saline for injection before being resuspended in the same to achieve a solution 80 mg/ml in concentration. 1 ul of this solution therefore contained 80 ug of TA and given the presumptive rat vitreal volume of about 55-60 μl, the estimated final concentration of TA in the eye is about 1.33 mg/ml similar to the concentration of 1 mg/ml usually used in patients clinically (47). All other procedures are as described above.

The results show that stem cell integration into the RGC layer was also accompanied by partial restoration of vision in these animals, as judged by recovery of the scotopic responses to low light in the electroretinogram (ERG). These results are shown in FIG. 7.

All documents cited above are incorporated herein by reference.

### REFERENCES

1. Takahashi et al., Molecular and Cellular Neuroscience 1998;12:340-348.
2. Qiu et al., Experimental Eye Research 2005;80:515-525.
3. Lund et al., PNAS 2001;98:9942-9947.
4. Lund et al., Stem Cells 2007;25:1089.
5. Dong et al, Neuroreport 2003;14 :143-146.
6. Canola et al., Invest. Ophthalmol. Vis. Sci. 2007;48:446-454.
7. Banin et al., Stem Cells 2006;24:246-257.
8. Gamm et al., PLoS ONE 2007;2.
9. Mizumoto et al., Cell Transplantation 2001;10:223-233.
10. Wojciechowski et al., Experimental Eye Research 2002;75:23-37.
11. Chacko et al., Vision Research 2003;43:937-946.
12. Young et al., Molecular and Cellular Neuroscience 2000;16:197-205.
13. Sakaguchi et al., Ann NY Acad Sci 2003;995:127-139.
14. MacLaren et al., Nature 2006;444:203-207.
15. Raymond et al., BMC Developmental Biology 2006;6:36.
16. Fischer et al., Nat Neurosci 2001;4:247-252.
17. Ooto et al., PNAS 2004;101:13654-13659.
18. Lawrence et al., Stem Cells 2007;2006-0724.
19. Lewis et al., Int Rev Cytol. 2003;230:263-290.
20. de Kozak et al., Ocul Immunol Inflamm 1997;5:89-94.
21. Thanos et al., Int J Dev Neurosci. 1993;11:671-680.
22. Roque et al., Invest. Ophthalmol. Vis. Sci. 1996;37:196-203.
23. Jones et al., Experimental Neurology 2003;182:399-411.

24. Morgenstern et al., Elsevier, 2002: 313-332.
25. Jones et al., J. Neurosci. 2003;23:9276-9288.
26. Inatani et al., Progress in Retinal and Eye Research 2002;21:429-447.
27. Selles-Navarro et al., Experimental Neurology 2001;167:282-289.
28. Cafferty et al., J. Neurosci. 2007;27:2176-2185.
29. Bradbury et al., Nature 2002;416:636-640.
30. Huang et al., Biochemical and Biophysical Research Communications 2006;349:963-968.
31. Kim et al., J Comp Neurol. 2006;497:182-198.
32. Limb et al., Invest. Ophthalmol. Vis. Sci. 2002;43:864-869.
33. Yanez-Munoz et al., Nat Med 2006;12:348-353.
34. Green et al., J Clin Pathol 1989;42:875-880.
35. Inatani et al., Invest. Ophthalmol. Vis. Sci. 2001;42:1930-1938.
36. Porrello et al., Current Eye Research 1986;5:981-993.
37. Zhang et al., Invest. Ophthalmol. Vis. Sci. 2003;44:1252-1261.
38. Uhlin-Hansen et al., J. Biol. Chem. 1988;263:2526-2531.
39. Jones et al., J. Neurosci. 2002;22:2792-2803.
40. Jones et al., J. Neurosci. 2002;22:4611-4624.
41. Gruter et al., Gene Ther 2005;12:942-947.
42. Rhodes et al., Journal of Anatomy 2004;204:33-48.
43. Berardi et al., Neuron 2004;44:905-908.
44. Pizzorusso et al., Science 2002;298:1248-1251.
45. Suzuki et al., Cell Transplantation 2007;16:493-503.
46. Lam et al., Investigative Ophthalmology Visual Science, 1999; 40: 2391-2397.
47. Gao et al., Vision Research, 2004; 44: 349-356.

1. A composition for improving stem cell migration and/or integration comprising:
a chondroitin sulphate proteoglycan (CSPG) inhibitory agent; and
an anti-inflammatory agent.

2. The composition of claim 1 further comprising stem cells.

3. (canceled)

4. The method of claim 14, wherein the individual has a CNS disorder.

5. The method of claim 4, wherein the CNS disorder is Parkinson's disease or spinal cord injury.

6. The method of claim 14, wherein the individual has a visual disorder.

7. The method of claim 6, wherein the visual disorder is selected from the group consisting of age-related macular degeneration, macular hole, proliferative diabetic retinopathy, proliferative vitreoretinopathy, retinal detachment, retinitis pigmentosa, retinopathy of prematurity, and general retinal dystrophies.

8. The composition of claim 1, wherein the chondroitin sulphate proteoglycan (CSPG) inhibitory agent is chondroitinase, a hyaluronidase, or a matrix metalloproteinase.

9. The composition of claim 8, wherein the CSPG inhibitory agent is chondroitinase ABC.

10. The composition of claim 1, wherein the anti-inflammatory agent is selected from the group consisting of: a steroidal anti-inflammatory agent, a non-steroidal anti-inflammatory agent, an immunosupressive, an antibody, a cytokine inhibitor, and combinations thereof.

11. The composition of claim 10, wherein the anti-inflammatory agent comprises prednisolone, indomethaeine, azathioprine and cyclosporine.

12. The method of claim 14, wherein each agent of the composition is delivered simultaneously.

13. (canceled)

14. A method for improving stem cell migration and/or integration in stem cell therapy of an individual comprising:
delivering an effective amount of a chondroitin sulphate proteoglycan (CSPG) inhibitory agent and an anti-inflammatory agent to the individual.

15. A pharmaceutically acceptable composition comprising a chondroitin sulphate proteoglycan (CSPCI) inhibitory agent and an anti-inflammatory agent, together with one or more pharmaceutically acceptable excipients.

16. (canceled)

17. A kit for improving stern cell migration and/or integration during stem cell therapy comprising;
a chondroitin sulphate proteoglycan (CSPG) inhibitory agent; and
an anti-inflammatory agent.

18. The pharmaceutical composition of claim 15 that additionally comprises a stem cell.

19. The kit of claim 17 that additionally comprises a stem cell.

20. The method of claim 14, wherein each agent of the composition is delivered sequentially.

21. The method of claim 14, wherein each agent of the composition is delivered separately.
